(19) United States
(12) Patent Application Publication
(10) Pub. No.: US 2025/0344656 A1
(43) Pub. Date: Nov. 13, 2025

(54) GRAIN TRACKING SYSTEM AND METHOD FOR USE IN AGRICULTURE SUPPLY CHAINS

(71) Applicant: Perdue AgriBusiness LLC, Salisbury, MD (US)

(72) Inventor: Perry AULIE, Salisbury, MD (US)

(21) Appl. No.: 19/277,126

(22) Filed: Jul. 22, 2025

Related U.S. Application Data
(63) Continuation of application No. 18/891,898, filed on Sep. 20, 2024.
(60) Provisional application No. 63/584,508, filed on Sep. 22, 2023.

(51) Int. Cl. A01H 1/04; C12Q 1/6895; G01N 33/00

(52) U.S. Cl. CPC A01H 1/04; C12Q 1/6895; G01N 33/0098; C12Q 2600/13; C12Q 2600/156

(57) ABSTRACT

Disclosed is a method for tracking an agricultural product variety such as a variety of a grain through a supply chain by identifying the agricultural product variety or the variety of the grain based on a distinguishable characteristic of a sample of the agricultural product or grain; wherein the distinguishable characteristic is phenotypic; and the distinguishable characteristic is (i) a seed characteristic of a plurality of seed in the sample; or (ii) a secondary characteristic of plants grown from the seed; or (iii) a combination thereof. Also disclosed is a method of making an agricultural product variety or variety of a grain identifiable by breeding to establish the distinguishable characteristic. Further disclosed is a taxonomy system for identifying an agricultural product variety, such as a grain variety, by defining a set of distinguishable structural features of the agricultural product variety.

This application claims priority to U.S. Provisional Patent application No. 63/584,508 filed Sep. 22, 2023, the entire contents of which are incorporated herein by reference.

### FIELD OF INVENTION

Disclosed herein are methods and systems for end to end tracking of products throughout a commodity supply chain, associated trackable products and methods for preparing the trackable products. In particular, systems, methods and products for tracking agricultural products are disclosed. More particularly, methods systems and products for tracking varieties of grains is disclosed.

### BACKGROUND

Tracking identity-preserved products, for example, agricultural products such as grains, in the global supply chain is essential to maintain the integrity of value-added products. For instance, tracking of agricultural products serves as a crucial function in at least three distinct scenarios: during product development or plant breeding, secondly, in guaranteeing quality differentiation, and thirdly, notably in the context of biotechnology, in aiding regulatory compliance by discerning between eligible and prohibited markets.

### SUMMARY OF INVENTION

Disclosed herein is a method for tracking an agricultural product variety such as a variety of a grain through a supply chain by identifying the agricultural product variety or the variety of the grain based on a distinguishable characteristic of a sample of the agricultural product or grain. The distinguishable characteristic is phenotypic and is (i) a seed characteristic of a plurality of seed in the sample; or (ii) a secondary characteristic of plants grown from the seed; or (iii) a combination thereof.

Also disclosed is a method of making an agricultural product variety such as a variety of a grain identifiable by a distinguishable characteristic by breeding the agricultural product variety or the variety of the grain to establish a distinguishable characteristic that is uniform, stable and heritable, wherein the distinguishable characteristic is (i) a seed characteristic of the agricultural product variety or the variety of the grain or (ii) a secondary characteristic of plants grown from a seed of the agricultural product variety or the variety of the grain; or (iii) a combination thereof.

Further disclosed is a taxonomy system for identifying an agricultural product variety such as a grain variety by defining a set of distinguishable structural features of the agricultural product variety, wherein the distinguishable structural features may be selected from one or more of seed shape, seed coat color, hilum color, plant seed luster, flower color, leaf shape, pubescence, pod appearance, plant shape, stem termination, bloom habit, leaf habitat, and combinations thereof.

### BRIEF DESCRIPTION OF FIGURES

Features and advantages of the present disclosure will be apparent from the following, more particular description of a preferred embodiment of the disclosure, as illustrated in the accompanying drawings wherein like reference numbers generally indicate identical, functionally similar, and/or structurally similar elements.

FIG. 1 is a flow chart showing components in a commodity grain system supply chain.

FIGS. 2A-2C illustrate structures and elements of a soybean that may be used as characteristics. FIG. 2A illustrates characteristics on soybean plants. FIG. 2B illustrates characteristics on soybean seed. FIG. 2C illustrates internal characteristics on soybean seed.

FIGS. 3A-3D illustrate structures and elements of the soybean seeds and pods that may be used as characteristics. FIG. 3A is a top view of a soybean seed. FIG. 3B is a side view of the soybean seed. FIG. 3C is a soybean pod. FIG. 3D is a view of a soybean seed as it would appear within a soybean pod.

FIGS. 4A-4C illustrate aspects of seed shape. FIG. 4A is a top view of a seed showing the length, FIG. 4B is a top view of a seed showing the width, and FIG. 4C is a side view of a seed showing the thickness.

FIG. 5 shows soybean seeds with patterned colors.

FIG. 6 shows a range of standardized hilum colors.

FIG. 7 shows soybean seeds with a combination of characteristics.

FIG. 8 illustrates leaf shapes.

FIG. 9 illustrates plant shapes.

FIG. 10 illustrates stem termination characteristics.

FIG. 11 illustrates an exemplary computer system.

### DETAILED DESCRIPTION

Features of the disclosure are discussed in detail below. In describing features, specific terminology is employed for the sake of clarity. However, this disclosure is not intended to be limited to the specific terminology so selected. While specific exemplary features are discussed, it should be understood that this is done for illustration purposes only. A person skilled in the relevant art will recognize that other components and configurations can be used without parting from the spirit and scope of the disclosure. All references cited herein are incorporated by reference as if each had been individually incorporated.

The terminology used herein is for the purpose of describing specific embodiments only and is not intended to be limiting of the invention.

As used herein, the singular forms “a,” “an,” and “the” include plural referents unless the context clearly dictates otherwise. The word “or” is the inclusive or and is used synonymously with “and/or” such that “A or B” includes embodiments having only A, embodiments having only B, and embodiments having both A and B. Nevertheless, the description should be read with the understanding that such combinations are entirely within the scope of the invention.

Disclosed herein are methods for tracking products throughout a commodity supply chain. The present disclosure describes methods focused on agricultural products. The term “agricultural products” as used herein refers to plant products in general, including ornamental plants, flowers and shrubs, and agricultural foodstuffs and plants including, for example, fruits, vegetables, nuts, grains, and other agricultural foodstuffs. Terms such as fruits, vegetables, nuts, grains are not intended to be limiting, but as general food categories. For example, most vegetables can be considered fruits of the plants from which they are grown, Agricultural foodstuffs refers to products grown for animal and/or human consumption used in the production of food products such as drinks, syrups, fermented products, flour, and others.

The term “agricultural product variety” as used herein refers to a product, e.g., a fruit, vegetable, nut, grain, and other agricultural foodstuff, with a particular property or set of properties. In general, the properties distinguish the agricultural product variety from other agricultural product variety or a commodity of the agricultural product. Properties can include source identification, use properties, growing properties, and other properties useful for the identification of a particular agricultural product variety.

Non-limiting examples of fruits include apples, oranges, apricots, lemons, limes, grapes, avocado, melons and squash (watermelon, cantaloupe, honeydew, pumpkins, zucchini, etc.) bananas, cherries, nectarines, pears, berries (blackberries, raspberries, strawberry, cranberry), grapefruit, plums, and mango. Non-limiting examples of vegetables include tomatoes, carrots, broccoli, beans (green beans, chickpeas, peas, etc,) kale, tubers (potatoes, sweet potatoes, radishes, turnips, yams), and cucumber. Nonlimiting examples of nuts include almonds, Brazil nuts, cashews, hazelnuts, macadamias, pecans, pine nuts, pistachios, walnuts, and peanuts.

Although the disclosed products, systems and methods can be applied to a wide variety of agricultural products, the disclosure is generally described for a grain. As used herein, the term “grain” refers to a species of a grain plant in general and includes seeds, crops and plants. Nonlimiting examples of grains include soybeans (or soy), corn, rye, rice, wheat, buckwheat, oats, millet, and barley. A “grain variety” or “variety of a grain” refers to a grain with a particular property or set of properties. In general, the properties distinguish the grain variety from other grain varieties or commodity grains. Properties can include source identification, use properties, growing properties, and other properties useful for the identification of a particular variety of a grain. Throughout this disclosure, soybean is described as a particular grain for illustrative purposes only, but this disclosure applies equally to other grains, as well as other agricultural foodstuffs.

In describing the disclosed features, it will be understood that several techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion.

### Grain Supply Chain

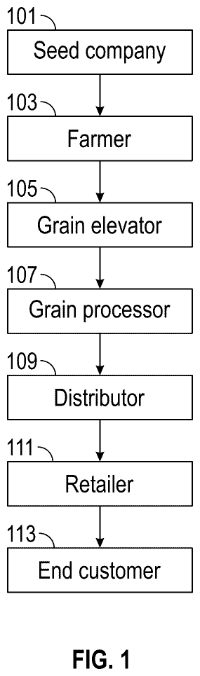

As shown in FIG. 1, the grain system supply chain involves a significant number of individuals that can benefit from the ability to track particular grain varieties. There are numerous reasons for tracking grains throughout the supply chain and different participants in the supply chain may have varying tracking interests. For example, seed producers 101 have an interest in assuring that their products are not copied, produced, or sold by competitors, or obtained (e.g., purchased or bred) by illegitimate means. Farmers 103 and distributors 109 have an interest in verifying the authenticity of purchased value differentiated grains. Grain elevators 105 and grain processors 107 have an interest in assuring grain identity and in adequately and efficiently segregating and storing grains. Segregating grains can include establishing distinct storage for a grain variety. Accordingly, the invention facilitates a readily applicable means to identify a grain variety and maintain separate storage from other varieties of the grain and from commodity grains. Additional benefits are outlined below.

The seed producer 101 performs seed breeding of value-added grain, which can include valuable and cost intensive steps including identifying Quantitative Trait Loci (QTL) for unique seed coat markings, validating the QTL is close to 100% heritable, and incorporating the seed coat marking QTL into a variety breeding program. The seed producer 101 can utilize the unique marking to track the grain within the seed breeding program and sell commercial grain varieties with the unique marking present enabling tracking for end-to-end grain tracking in agriculture commodity supply chains.

The farmer 103 purchases a commercial variety of grain with the unique marking from seed producer 101 and plants the variety and provides agronomic management through the growing season. The present invention allows the farmer 103 to be certain that seed purchased is the proper seed variety.

The grain elevator 105 purchases harvested grain from the farmer 103 with the unique marking, assuring that it is the represented variety of grain. The disclosed method and system further allows the grain elevator 105 to segregate the unique grains in storage to maintain identity preservation and sell and deliver the identity preserved grain to customers.

The grain processor 107 purchases harvested grain from the grain elevator 105 with the unique marking. The marking allows the grain processor 107 to authenticate the value-added grain with the end-to-end tracking system, segregate the unique grain in storage tanks to maintain identify preservation, perform value added grain processing on the identity preserved grains, segregate end products of the grain processing, and confidently market the value-added end products.

The distributor 109 purchases end products from the grain processor 107. The marking allows the distributor 109 to produce consumer-packaged goods using the properly identified end products purchased from the grain processor and sells the authenticated consumer-packaged goods to a retailer 111.

The retailer 111 purchases consumer-packaged goods from the distributor 109 and can authenticate value added consumer products for sale for end consumers 113. End consumer 113 can then confidently purchase consumer-packaged goods from the retailer 111 and use the consumer-packaged goods.

Current traceability initiatives in the grain industry do not offer comprehensive end-to-end solutions for identifying, monitoring and tracking grains through the supply chain connecting the plant breeder to the end-user. Instead, they rely on introducing foreign materials to the grain after it has already entered the supply chain (see, e.g., U.S. Pat. No. 8,810,406B2). Other methods involve printing barcodes on grains using starch-based tracers. These approaches do not possess the necessary durability to withstand the abrasive grain handling system that can occur at multiple points in the supply chain. Furthermore, the implementation of these tracking devices requires a physical intervention, creating a noteworthy vulnerability to achieving a reliable end to end tracking of grains. In summary, exemplary limitations of the current solutions include, but are not limited to:
Inability to establish end-to-end connectivity from plant breeder to end user.
Lack of durability for the abrasive commodity supply chain.
Introduce foreign material into the food supply chain.
Require individuals, e.g., farmers to perform incremental tasks which are not practical during the hectic harvest period.
High cost making them prohibitive to adopt at scale to track the billions of pounds of bulk commodity grains produced annually.

Other methods of identifying grain varieties, for example, analysis of internal seed structures, can require destructive testing such as DNA testing, and it is less discernible whether an entire sample of grain is of the same origin. Destructive testing can also be time consuming and require outside testing rather than on-site identification.

This disclosure describes establishing a distinguishable characteristic (also referred to herein as a mark or marker) in a grain variety, using the distinguishable characteristic for end to end grain tracking in agricultural supply chains, and a taxonomy system for hierarchical classification of the marked grains for end to end grain tracking in agricultural commodity supply chains and other purposes. The distinguishable characteristic may be a single characteristic or a set of characteristics which, taken in total, identify the grain variety.

Many grains are fungible commodities. For example, varieties of soy or soybeans, are largely interchangeable and a farmer or consumer would have no preference of one variety over another. However, seed producers spend time and effort developing particular seed and grain varieties using traditional methods, biotechnology methods and others to produce grains with value differentiating characteristics such as, for example, source, improved disease resistance, antioxidant content, nutrient content, yield, growth processing characteristics, utilization characteristics, environmental adaptation ability. There are a number of reasons to effectively track or identify grains to determine the grain variety including, for example:
Protection from unauthorized distribution of grain and seeds.
Quality and safety assurance.
Verifying the identity of a purchased grain.
Monitoring location of grain shipments.
Enforcing market restrictions where grains are not permitted in certain regions or markets due to regulatory constraints or commercial agreements. (For example, preventing importation of varieties of grains not permitted in certain jurisdictions such as restrictions on importation of genetically modified in some European countries.)
Identification for product recall purposes.

### Distinguishable Characteristic

A distinguishable characteristic provides the means for differentiating a grain variety from other varieties of the grain and/or from commodity grains. A distinguishable characteristic is based on attributes of particular characteristics of the grain variety The term “attributes” is generally used herein to identify specific traits of a characteristic. For example, a plant characteristic may be flower color and the attributes would be individual colors such as white, yellow, purple, etc. Identification of the grain variety can be made by identifying the distinguishable characteristic which may be an attribute of a single characteristic or a set of attributes of characteristics.

A plant or seed may include several sets of distinguishable characteristics to provide differing tracking methods or purposes. For example, as described further below, a seed producer may be identified with a particular seed color to provide origin information. This could be used by the seed producer to track its grain through a commerce chain, or by a farmer to verify that a grain sample, e.g., a bag of grain, is from a particular producer or breeder and separate the variety of grains from other grains made by a different producer or breeder. Such a distinguishable characteristic could also be used by a breeder or producer to verify that a party in possession of the seed obtained it through legitimate channels. The same seed could also include a second distinguishable characteristic to indicate some other value differentiator for a grain. For example, characteristics, such as seed shape, seed coat color, hilum color, plant seed luster, and/or any secondary characteristic, taken alone or in combination, could indicate properties such as disease resistance; antioxidant content; nutrient content; yield, growth, and crop management character; processing and utilization; environmental adaptation; and/or regulatory status. It may be desirable to track a grain variety with any one or more of these properties.

In order to utilize a characteristic as set forth in this disclosure, the characteristic must be phenotypic and have a value of heritability close to 100%. This ensures that every year, regardless of the environmental conditions, the markings will be uniform, stable, and present. When the heritability has no environmental variance, then all the phenotypic variance is genetic variance. This heritability ensures the unique appearance of the characteristic can be monitored on all desired grains within the breeding program and subsequently at each step of the commodity grain supply chain.

In embodiments, the characteristics are perceptible to the human eye. In embodiments, the characteristic may be detected using a scanner or image generating device. In embodiments, the characteristic may be detected using advanced sensor technologies such as thermal imaging, multispectral imaging, hyperspectral imaging, and LiDAR. The image generating device or sensor technology can be used in conjunction with a computer system for analyzing the characteristic or characteristics and identifying the variety of the grain.

The computer system can, for example, execute image recognition algorithms, using inputs from the cameras, sensors, or other input mechanisms, and outputting likely object. Such algorithms can, for example, utilize a neural network trained on images of seeds/plants having known characteristics, where during execution the neural network the system analyzes newly received images, compares attributes of those images to known grain varieties, and outputs a likely grain variety to which the scanned seeds/plants belong.

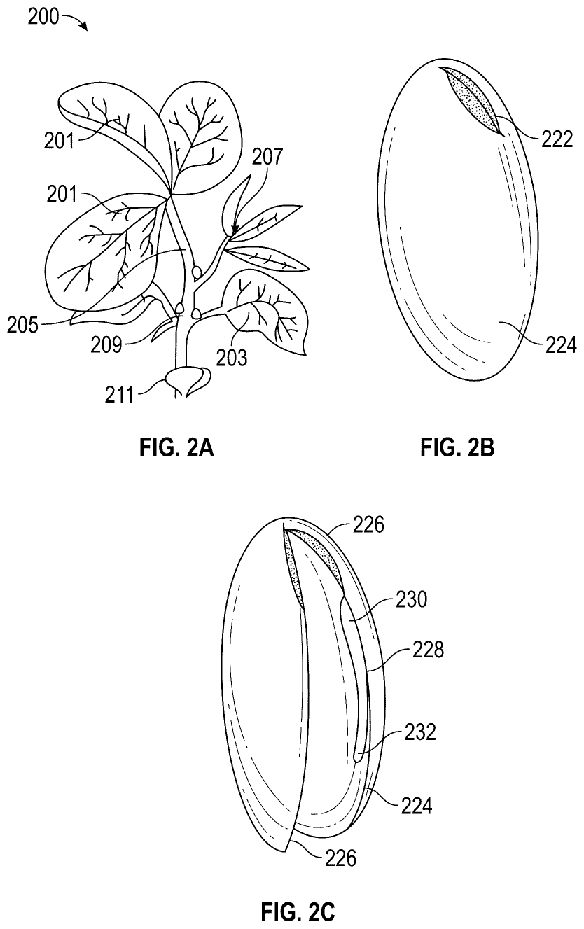

Distinguishable characteristics can include characteristics of the grain seed, which may be external or internal, and the grain plant. Characteristics of the grain plant are sometimes referred to herein as secondary characteristics. FIG. 2 illustrates some exemplary characteristics of soybean that may be used as a distinguishable characteristic or included in a set of characteristics to define a distinguishable characteristic. Although soybean is used as a specific exemplary grain in the present disclosure, the disclosure is not intended to be limited to soybean nor to grains. FIG. 2A illustrates characteristics of a soybean plant 200. Plant characteristics, also referred to herein as secondary characteristics, include for example, leaf structure (e.g., trifoilate leaf 201 or Unifoliate leaf 203), petiole 205, attributes of a growing point 207, shape of auxiliary buds 209, and structural features of plant cotyledon 211. Distinguishable characteristics can include color, color pattern, shape and other attributes of these parts. Additional characteristics are described elsewhere herein and identifiable by persons skilled in the art.

FIG. 2B and FIG. 2C illustrate some external characteristics of a soybean seed 220. External characteristics of a soybean seed (also referred to as seed coat characteristics) include the hilum 202, seed coat 224, and cotyledon 226. Internal structures of the seed 220 can include parts of the embryo such as the hypocotyl 228, the radicle 230, and the epicotyl 232. Distinguishable characteristics can include color, color pattern, shape and other attributes of these parts. Additional characteristics are described elsewhere herein and identifiable by persons skilled in the art.

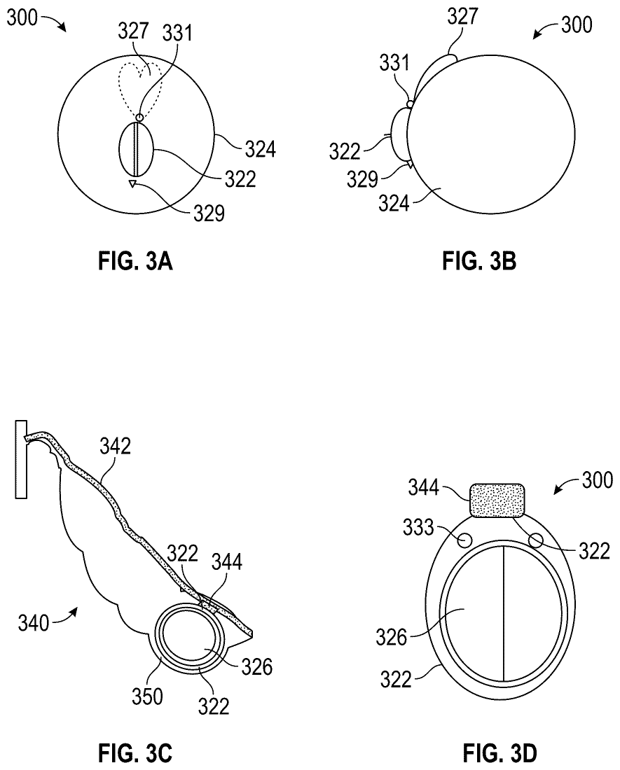

FIG. 3 illustrates additional structures and elements of the soybean seeds 320 and pods 340 that may be used as characteristics. FIG. 3A and FIG. 3B are top and side views, respectively, of a soybean seed 320. FIG. 3D is a view of a soybean seed as it would appear within or shorty after removal from a soybean pod. Characteristics of the soybean seed illustrated in FIG. 3 include hilum 322, seed coat 324, cotyledon 326, hypocotyl/radicle 327, raphe 329, micropyle 331, vascular bundle 333, As shown in FIG. 3A, the hilum can include additional structural features such a central fissure 335. FIG. 3A and FIG. 3B further illustrate the hypocotyl/radicle 327 radius or shape. FIG. 3C is a soybean pod 340, shown in an open configuration with an attached seed 350, and identifying the arrangement of the main vein 342 of the soybean pod 340, and the funiculus 344, connecting the seed to the pod 340, which forms the hilum on the seed when removed from the pod. Distinguishable characteristics can include color, color pattern, shape and other attributes of these parts. Additional characteristics are described elsewhere herein and identifiable by persons skilled in the art.

While the seed characteristics are the primary characteristics that make up the distinguishable characteristic. In some embodiments, additional secondary characteristics may be used in place of or in addition to the primary characteristics. These secondary characteristics may encompass characteristics of the plant such as flower color, leaf shape, pubescence, mature pod appearance, bloom habit, and others. (See, e.g., FIG. 2A.). Integrating secondary markings can be particularly useful for tracking purposes in the agriculture supply chain. For example, some secondary markings are key when the seeds are not visible, such as during the growing season when only the vegetative parts of the plant are above the ground. Distinguishable characteristics can include color, color pattern, shape and other attributes of these parts. Additional characteristics and attributes are described elsewhere herein and identifiable by persons skilled in the art.

Specific attributes can be unique, newly developed attributes or existing attributes of characteristics. For example, a seed may have a unique color (as defined further below) that identifies the source or producer of the seed. Patterns, combinations of colors, or a combination of colors and patterns can be similarly distinguishing. The development of these unique attributes has the further advantage as possibly being designated for additional protection, for example, as a trademark or as trade dress. Typically, when an existing color or pattern is used it can be a distinguishable characteristic when considered in combination with other characteristics.

### Seed Characteristics

In embodiments, the distinguishable characteristic is a seed characteristic. In further embodiments seed coat characteristics are regarded as the primary identifiers.

Those skilled in the art understand that in some instances, seed characteristics may be correlated to particular properties of the grain. For example, the seed coat color may be correlated with properties such as:
Disease Resistance: Some studies suggest that seed coat color is associated with disease resistance. Darker-colored seed coats might offer better protection against certain pathogens due to the presence of compounds like tannins that have antifungal properties.
Antioxidant Content: Seed coat color can be linked to antioxidant content. Darker-colored seed coats often contain higher levels of compounds like anthocyanins.
Nutrient Content: Seed coat color can also reflect the presence of certain nutrients. For instance, certain colors might indicate higher levels of specific vitamins, minerals, or phytochemicals.
Yield and Growth: Seed coat color could potentially influence growth rates and yield. Some research suggests that seed coat color might correlate with traits related to plant growth and productivity.
Processing and Utilization: Seed coat color can affect the processing and utilization of soybeans. For example, certain colors might be preferred for specific processing methods or end products.
Environmental Adaptation: Seed coat color might be linked to environmental adaptation. Distinct colors could have evolved in response to specific ecological conditions, which could influence the soybean's ability to thrive in certain environments.

However, according to the present disclosure, the characteristics, such as seed coat color are disassociated with these properties and other factors that impact the utility of the grains. Alternatively, while seed coat color may be an indicator of a particular aspect of the grain, e.g., antioxidant content, that characteristic can be combined with another characteristic, e.g., hilum color, such that the plurality of individual features identify the variety of the grain.

As noted above, seed characteristics include, for example, seed shape, seed coat color, hilum color, and plant seed luster. Seed characteristics or attributes can be unique, newly developed attributes or existing attributes of characteristics. Other characteristics, attributes, features and markings alone or in combination with other characteristics, can be used as distinguishable characteristics.

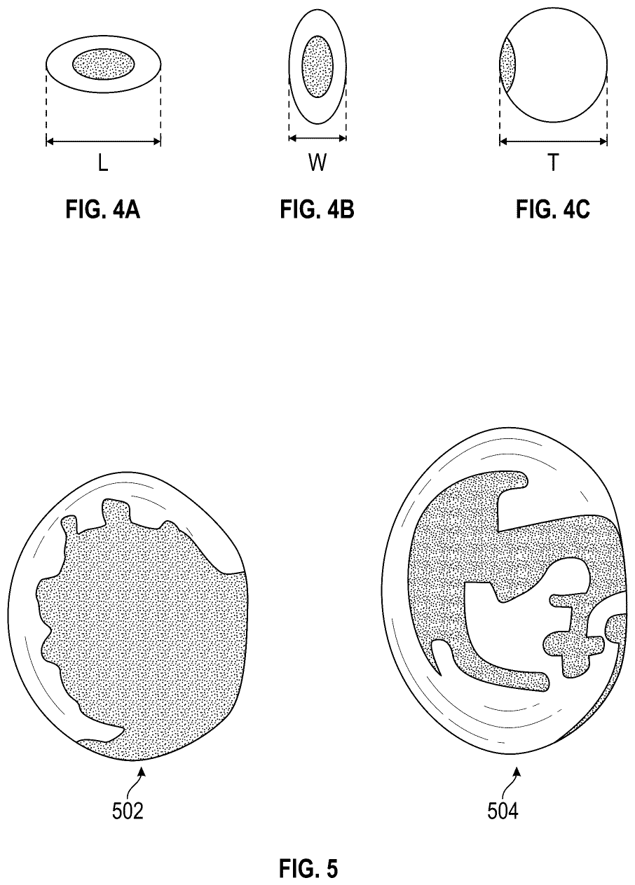

FIG. 4 illustrates aspects of seed shape, including the length L (FIG. 4A), width W (FIG. 4B), and the thickness T (FIG. 4C). Attributes based on seed shape can include, for example, spherical rounded, (wherein the length/width, length/thickness, and thickness/width ratios ≤1.2); spherical flattened (wherein the length/width ratio >1.2 and the length/thickness ratio <1.2), elongate (wherein the length/thickness ratio >1.2 and the thickness/width ratio <1.2). Other attributes can be defined.

Seed coat color attributes can include colors, color patterns, a combinations of colors, or a combination of colors and patterns. For example, existing colors of soybean seeds can include, yellow, black, brown, dark brown, red-brown, buff, and green. Colors can be uniform, combinations of colors (e.g. dichromate seeds made up of two colors) or exist in color patterns. Seed coat color is used herein to refer to color(s) and/or color pattern. FIG. 5 shows a soybean seed with a saddled color pattern 502 and a soybean seed with a pattern of black dots or concentric rings 504.

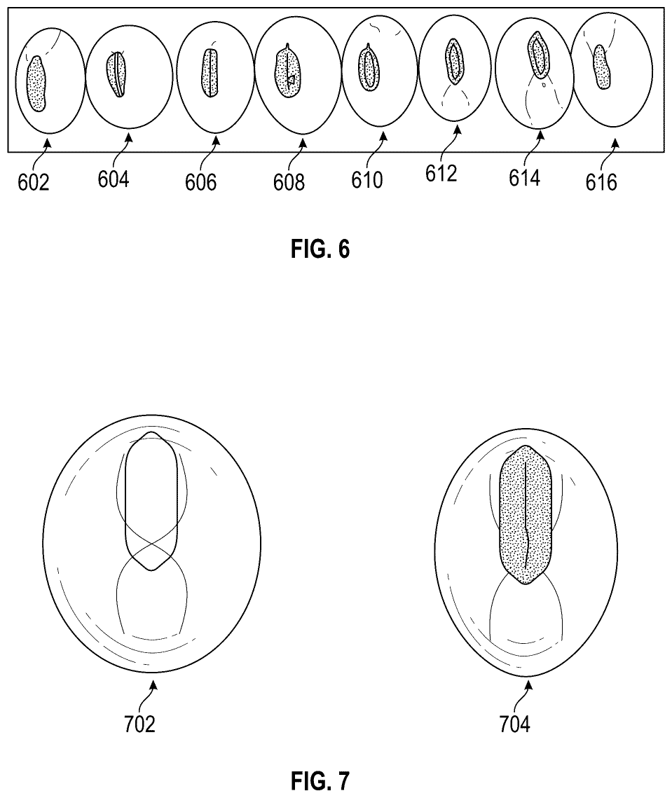

FIG. 6 show a range of standardized hilum colors that can be attributes of the hilum color characteristic-black hilum 602, imperfect black hilum 604, grey hilum 606, dark brown hilum 608, brown hilum 610, light brown hilum 612, imperfect yellow hilum 614, and yellow hilum 616.

Plant seed luster attributes can include dull, mudfilm, mud-free-film and lustrous.

A distinguishable characteristic can be based on a combination of characteristics. For example, FIG. 7 shows a first soybean seed 702 with a yellow coat and yellow hilum and a second soybean seed 704 with a yellow coat and black hilum. While the soybean seeds in FIG. 7 are based on a combination of two characteristics to illustrate a distinguishable characteristic, more than two characteristics may make up the distinguishable characteristic.

For agricultural products other than grains, for example fruits and vegetables, additional characteristics can be used as distinguishable characteristics. Nonlimiting examples of additional characteristics can include fruit shapes, fruit colors (including, as described above colors, color patterns, a combinations of colors, or a combination of colors and patterns), and fruit texture, (e.g., rough, smooth ridged, and patterns of textures).

### Secondary Characteristics

As noted above, secondary characteristics may be used in place of or in combination with the primary characteristics to identify a variety of a grain. Secondary characteristics may encompass characteristics of the plant. Secondary characteristics can thus be identifiers such as flower color, leaf shape, leaf configuration, pubescence, pod appearance, plant shape, stem termination, bloom habit, leaf habitat and others. A distinguishable characteristic can be based on a combination of characteristics.

As with seed characteristics, attributes can be unique, newly developed attributes or existing attributes of characteristics. For example, a flower or plant may have a unique color or pigmentation (including, as described above colors, color patterns, a combinations of colors, or a combination of colors and patterns) which identifies the source or producer of the plant. Typically, when an existing color or pattern is present, it can be a distinguishable characteristic when considered in combination with other characteristics. Flower color attributes can be, for example, white, purple, and others. Plant pigmentation is another color characteristic that can be utilized. Pigmentation can include not only visible color of the plant or leaves, but also incorporation of pigments detectable using infrared sensors, ultraviolet sensors, or fluorescence sensors. These pigmentation characteristics are particularly useful when using sensing technologies such as thermal imaging, multispectral imaging, hyperspectral imaging, and LiDAR, especially in the sphere of crop management as described further below.

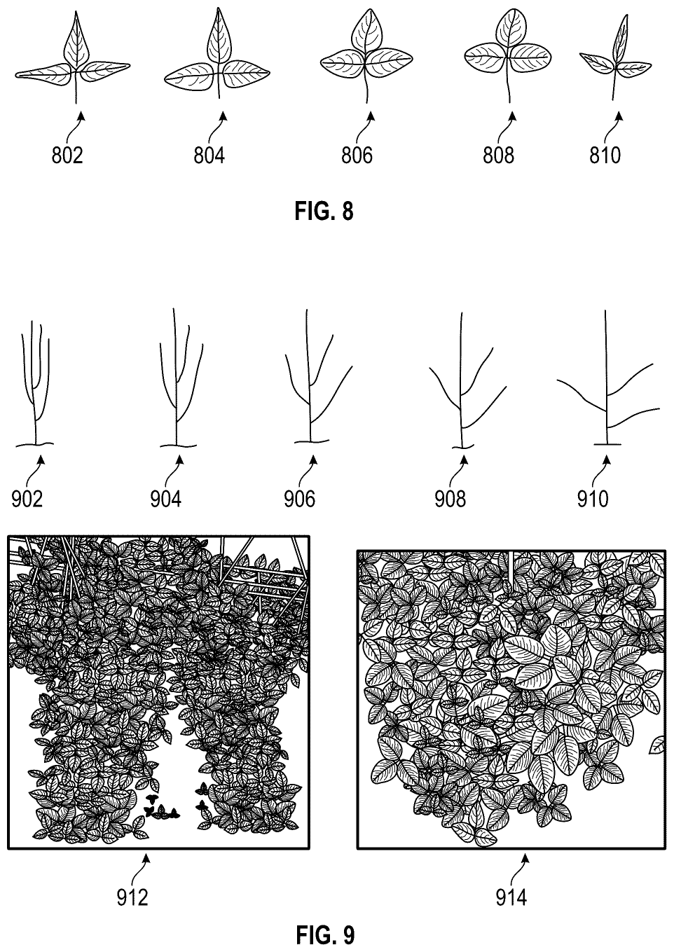

Secondary characteristics can be associated with the leafy portion of the plant. For example, FIG. 2 shows a trifoliate growth pattern 201 and a unifoliate growth pattern 203. As shown in FIG. 8, leaf shape can be lanceolate 802, triangular 804, pointed ovate 806, rounded ovate 808 or linear 810. Other growth pattens and leaf shapes may also be displayed.

Pod appearance can be, for example, brown, dark brown and black.

Plant shape can show several different attributes. For example, as illustrated in FIG. 9, plant shape can be erect 902, semi-erect 904, intermediate 906, semi-prostrate 908, prostrate 910, wild 912, semi-wild 914, and combinations thereof.

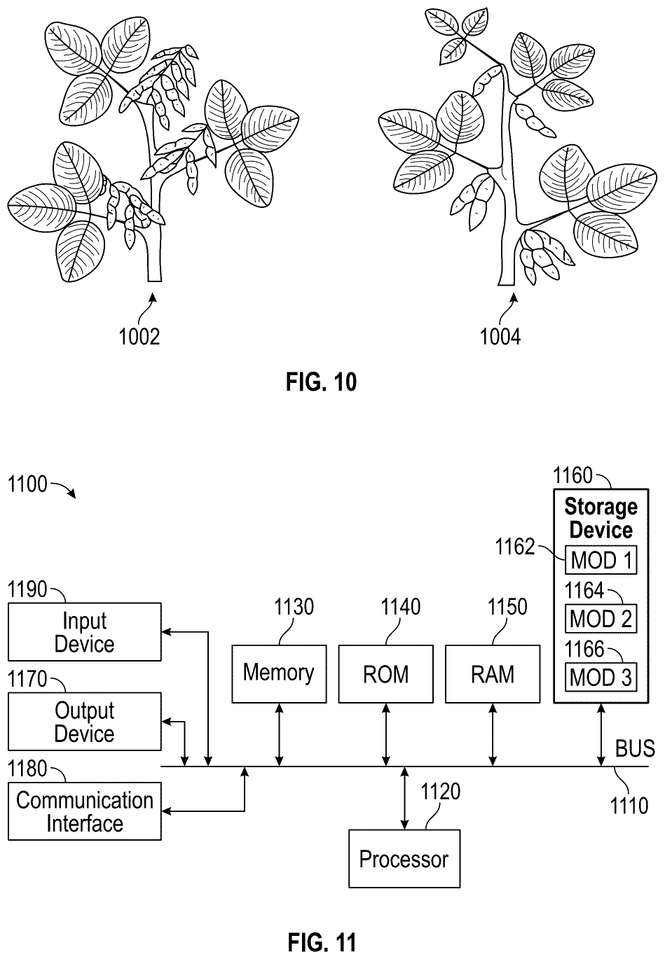

As illustrated in FIG. 10, stem termination attributes can be, for example, determinate 1002 or indeterminate 1004.

### Value Differentiation

In marketing of agricultural products and grains, particularly varieties of grain developed to exhibit specific properties, it can be important to distinguish grain qualities, ownership, and other vital factors. Value differentiation characteristics are a property of a grain that makes it unique in the marketplace or designates an aspect of the grain variety distinguishing it from other grain varieties. Value differentiation arises when a supply chain actor is interested in a particular grain variety rather than a commodity grain. The value may be differentiated as more economically valuable than a commodity grain, for example a grain developed to grow under particular conditions or for a particular purpose. While value may be measured by cost, a value differentiated grain variety need not be more expensive to be more valuable. For example, a grain variety bred to have desirable properties for use as a cover crop may be less expensive than a commodity grain, while a grain variety with high nutrient content may be more expensive than a commodity grain. The tracking system disclosed herein provides the ability to tell the difference.

Identifying the variety of the grain can be important throughout the supply chain. For example, a seed company may want to track a grain variety through the supply chain to assure that the grain is only in the possession of purchasers licensed to grow or use the grain variety. A farmer may pay a premium for a variety of grain that has increased disease resistance. Grain elevators and grain processors have an interest in assuring grain identity and in adequately and efficiently segregating and storing grains with value added properties. The invention facilitates a readily applicable means to identify a grain variety with certain value differentiators and maintain separate storage from other varieties of the grain with different value differentiators. The invention also provides a means for segregation of a grain variety with a value differentiator from commodity grains. A processor may also purchase a grain for a particular production purpose, for example making flour as opposed to having a source for oil extraction. Without being able to readily identify the variety of grain, there may be no way of knowing whether the grain sold or purchased is, in fact, the variety they purport to be.

As described above, current methods of grain differentiation, and thus value differentiation, are inadequate by, for example, relying on the addition of an external material, which could be added to any grain, or grain markings that are insufficiently durable. Both of these examples also suffer from the drawback of being external to the grain itself, rather than being a heritable trait that can effectively track grain not only through the supply chain, but across generation of growth.

Value differentiation may be based on seed source or supplier, a utilization characteristic, a crop management characteristic, or other beneficial properties of the grain variety such as disease resistance, drought resistance, pest resistance, herbicide resistance, antioxidant content, nutrient content, improved yield, improved growth, a processing characteristic, or an environmental adaptation ability.

A utilization characteristic can be a property related to use or origin. For example, it may be important to differentiate a grain varieties intended for human consumption, intended for animal consumption, intended for use as an oil source, and/or intended for use as a solid food source. Because there may be regulatory restrictions related to the origin of the grain, it can also be important to determine whether the grain is of a genetically modified origin, a non-genetically modified origin, from a geographic origin and the like.

Value differentiation may also be related to crop management; e.g., a particular grain variety may be designed to grow in particular environmental conditions (e.g., arid vs. temperate), or resistant to particular diseases. Crop management can include, for example, crop segregation, fertilization parameters (e.g., rate of application and identity of fertilizers), herbicidal parameters (e.g., rate of application and identity of herbicide), harvest timing, irrigation timing, and the like. Crop management characteristics of the grain can thus include or be indicative of agronomic/soil management, improved yield, improved growth, a processing characteristic, a utilization characteristic, an environmental adaptation ability, and combinations thereof.

### Taxonomy System

Methods of the present disclosure allow for the creation of a taxonomy system designed for the categorization of grains. The taxonomy system structure, rules, and methodologies are valuable when implementing the marking system on a large scale. It establishes critical connections between the unique markings, grain qualities, grain ownership, geographic origin, and regulatory status, and other vital factors for stakeholders.

The taxonomy system can employ a hierarchical approach to the markings in which not all characteristics are of equal significance or relevance; some markings might carry more importance than others when some assessing grain quality or ownership. Therefore, it assigns distinct levels of importance or “weights” to these markings based on their relevance and significance.

By incorporating a hierarchical approach, the taxonomy system enhances the efficiency of the classification process. It does so by giving greater consideration to the more critical markings and less emphasis on those with lower significance. This not only accelerates the classification procedure but also serves as a safeguard against economic risks stemming from potential misclassifications. In the taxonomy system of the present disclosure, certain markings on grains are considered more important, and the taxonomy system focuses more on them to ensure accurate and efficient grain classification while reducing the chances of costly mistakes.

Efficient and cost-effective classification within this taxonomy system fosters widespread adoption by saving both time and resources. Visual characteristics like seed coat color and shape, akin to the grain industry's approach to distinguishing wheat, corn, and soybeans, serve as valuable classification criteria. This taxonomy system streamlines the identification of traceable grain varieties within the supply chain, distinct from commodity grains. Moreover, it has the potential to offer even more comprehensive and informative classifications.

For example, a unique seed color may be used to indicate a seed source-a high level but non-specific indicator or variety—while other characteristics such as seed shape, color pattern, hilum color, and combinations thereof may be indicative of particular varieties of grain from that source. Grain from a different source could have a different seed color but otherwise have the same combination of seed shape, color pattern, and hilum color, but have very different properties or value differentiators.

In practical terms, the taxonomy system's effectiveness acknowledges the limitations present across the supply chain, particularly if a universal system is utilized. For instance, seed breeders possess greater resources and time for precise seed classification compared to commercial farmers or grain handlers. Consequently, the taxonomy system, when utilized by a seed breeder, provides in-depth information. Conversely, a grain farmer might require simpler knowledge, such as identifying source or value differentiated seeds that need separate storage and use from commodity grains.

These concepts include a hierarchical classification of markings, offering stakeholders a structured insight into value differentiated grains. Markings can be assigned to categorize value differentiated grains by company, distinct features, geographic origin, regulatory status, or other pertinent criteria. This taxonomy tool mitigates confusion among stakeholders and supply chain participants.

A taxonomy system integrated with unique marks, when implemented within a supply chain, functions as identifiers at every stage, ensuring authenticity, origin, and intellectual property protection. By combining unique marks with a structured classification system, supply chain traceability is enhanced. It categorizes products, aiding in monitoring and verifying their journey, origins, and quality. This, in turn, fosters transparency and trust among stakeholders.

By way of example, consider a marking system based on characteristics with variations in each such as:
Flower color (white or purple)
Leaf shape (needle, oval, elliptical, linear)
Plant shape (wild, semi-wild)
Seed coat color (yellow, green, black, brown, dichromate)
Pod color (black, brown)
Seed luster (Mudfilm, mud-free film, lustrous)

This system of six characteristics with a total of 18 variations allows for the potential to track nearly 500 distinct grain varieties.

### Identification of Grain Variety

The distinguishable characteristic of a grain sample to identify a variety of a grain can be detected in a number of ways. The simplest methodology for identification is visual assessment. This is particularly useful in that it allows for rapid analysis and determination of grain variety at any level in the supply chain (see FIG. 1). A method similar to visual assessment could involve use a scanning technology, including a handheld scanner, to assess the distinguishable characteristics. Software, which could be part of the scanner or on an independent platform, could then provide a more automated system to identify the variety of the grain. Use of a scanner or other imaging system has the further advantage of possibly identifying more subtle characteristics, for example a ridge or a bump on a seed, that may be more difficult to discern or differentiate visually. An example may be for visualizing a pattern on the surface of a seed caused by the Hypocotyl/radicle shape within the seed. These methods are particularly useful in identifying relatively small samples of grain, such as a bag of seed.

Embodiment of the present disclosure are also directed to a computerized system and method of grading and authenticating grains utilizing digital imaging devices and processes to provide an objective, standardized, consistent high-resolution grading. The disclosure minimizes the subjectivity present in the human grading process and overcomes the inherent limitations of the human eye. For example, the frequencies of light visible to the human eye are limited to a defined bandwidth, whereas sensors can capture light outside that bandwidth (i.e., infrared or ultraviolet light). Furthermore, these sensors can have a degree of detail beyond that which can be captured by a human eye.

More intricate scanning and sensing systems can also be utilized to identify characteristics. These could include thermal imaging, multispectral imaging, hyperspectral imaging, and LiDAR. The imaging or sensing systems can be used to collect data and that data introduced to software to provide an automated system to identify the variety of the grain. With a proper selection of characteristics, these technologies can also be incorporated into grain processing equipment within the supply chain. For example, these technologies can also be incorporated farm equipment (tractors, harvesters, combines, etc.). When used with farm equipment, identification of the grain could be integrated with crop management systems, such as sprayers or irrigation systems, care for the growing crop by, for example, spraying herbicides, watering crops, or timing cultivation. Additionally, these technologies can be incorporated into aircraft to survey larger areas of land and are thus particularly useful for crop management. For example, as planes or other aircraft fly over land with cameras (and/or other sensors), data from the cameras can be provided to a computer system configured with plant identification software capable of performing the methods disclosed herein. As the aircraft captures data/images, that information can be analyzed by the computer system to identify the type of crop, plants, etc., which are being detected. When grain is transported to subsequent stages of processing, such as the grain elevator 105 (FIG. 1) for example, the grain can be sampled and these technologies can be incorporated into the sample processing system or into grain conveyance equipment, such as augers and conveyers. This can be further use to segregate grain varieties and maintain the integrity of supplies of grains with different properties.

One exemplary technical improvement of such a computer system can include improved accuracy. For example, by using a combination of different types of images (e.g., thermal imaging+LiDAR), the system can identify (e.g., via the neural network) correlations between the different characteristics which would otherwise not be recognized. When reviewing new images using those different types of images, the system can use those correlations to produce a level of recognition which is superior to using any individual imaging type alone. Another exemplary technical improvement can be in the form of immediate adjustments to detected conditions. For example, as a farmer uses equipment configured with sensors as disclosed herein, the sensor data can be immediately processed, and the farmer's equipment can be immediately adjusted (i.e., in real-time) based on the system's analysis of the sensor data to provide what the detected crops or plants need (e.g., more water, a different concentration of herbicide, etc.).

For example, the cameras/sensors can be placed on the tractor, harvester, etc., such that as the vehicle passes over the seeds or plants images/data is captured, then provided to the computer system for further analysis. In some configurations, the output of the detection system can be used to determine the further actions of the vehicle based on details detected from the images/data. For example, the images/data of a crop may result in the system identifying one or more of categories such as (but not limited to) the type of crop, the age of the crop, the quality of the soil, a water level of the soil, amount of non-desired plants (e.g., weeds), amount of non-desired pests (e.g., bugs that may harm the plants). Based on this information, the system can determine how to treat or otherwise interact with the crop. Such determinations can, for example, be generated using a database which identifies a current level of irrigation based on crop type, crop age, weeks until harvest, intended crop use (e.g., consumption v. seed), etc. Computations to determine the use of herbicide, pesticide, or other crop management can be made using similar databases configured to support a given category of care. In other configurations, the determination could be generated using an Al algorithm (e.g., neural network) configured to receive sensor inputs, analyze the data, then output an action (e.g., water the crops with a given amount of water, apply a prescribed amount of herbicide/pesticide, etc.). Using such a system, the farmer may drive over the crop and the farm equipment, working in tandem with the disclosed system, can automatically deploy the treatment needed for a given crop. Moreover, the treatment may vary as the farmer moves from location to location. For example, in one acre the system may determine that the crop needs to receive 0.5 inches (1.27 cm) of water/square foot (0.0929 m²), whereas in another acre the system may determine that the crop does not need additional water at this time. In another example, the system may detect a first strain of a crop and determine a first rate at which to spread fertilizer, then upon moving to a different portion of the field detect a distinct/2nd strain of the crop and determine a second, distinct rate at which to spread the fertilizer. Such changes can happen automatically as the farm equipment navigates the farm.

With reference to FIG. 11, an exemplary system includes a computing device 1100 (such as a general-purpose computing device), including a processing unit (CPU or processor) 1120 and a system bus 1110 that couples various system components including the system memory 1130 such as read-only memory (ROM) 1140 and random access memory (RAM) 1150 to the processor 1120. The computing device 1100 can include a cache of high-speed memory connected directly with, in close proximity to, or integrated as part of the processor 1120. The computing device 1100 copies data from the system memory 1130 and/or the storage device 1160 to the cache for quick access by the processor 1120. In this way, the cache provides a performance boost that avoids processor 1120 delays while waiting for data. These and other modules can control or be configured to control the processor 1120 to perform various actions. Other system memory 1130 may be available for use as well. The system memory 1130 can include multiple different types of memory with different performance characteristics. It can be appreciated that the disclosure may operate on a computing device 1100 with more than one processor 1120 or on a group or cluster of computing devices networked together to provide greater processing capability. The processor 1120 can include any general-purpose processor and a hardware module or software module, such as module 1 1162, module 2 1164, and module 3 1166 stored in storage device 1160, configured to control the processor 1120 as well as a special-purpose processor where software instructions are incorporated into the actual processor design. The processor 1120 may essentially be a completely self-contained computing system, containing multiple cores or processors, a bus, memory controller, cache, etc. A multi-core processor may be symmetric or asymmetric.

The system bus 1110 may be any of several types of bus structures including a memory bus or memory controller, a peripheral bus, and a local bus using any of a variety of bus architectures. A basic input/output (BIOS) stored in memory ROM 1140 or the like, may provide the basic routine that helps to transfer information between elements within the computing device 1100, such as during start-up. The computing device 1100 further includes storage devices 1160 such as a hard disk drive, a magnetic disk drive, an optical disk drive, tape drive or the like. The storage device 1160 can include software modules 1162, 1164, 1166 for controlling the processor 1120. Other hardware or software modules are contemplated. The storage device 1160 is connected to the system bus 1110 by a drive interface. The drives and the associated computer-readable storage media provide nonvolatile storage of computer-readable instructions, data structures, program modules and other data for the computing device 1100. In one aspect, a hardware module that performs a particular function includes the software component stored in a tangible computer-readable storage medium in connection with the necessary hardware components, such as the processor 1120, system bus 1110, output device 1170 (such as a display or speaker), and so forth, to carry out the function. In another aspect, the system can use a processor and computer-readable storage medium to store instructions which, when executed by a processor (e.g., one or more processors), cause the processor to perform a method or other specific actions. The basic components and appropriate variations are contemplated depending on the type of device, such as whether the computing device 1100 is a small, handheld computing device, a desktop computer, or a computer server.

Although the exemplary embodiment described herein employs the storage device 1160 (such as a hard disk), other types of computer-readable media which can store data that are accessible by a computer, such as magnetic cassettes, flash memory cards, digital versatile disks, cartridges, random access memories (RAMs) 1150, and read-only memory (ROM) 1140, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 1100, an input device 1190 represents any number of input mechanisms, such as a microphone for speech, a touch-sensitive screen for gesture or graphical input, keyboard, mouse, motion input, speech and so forth. An output device 1170 can also be one or more of a number of output mechanisms known to those of skill in the art. In some instances, multimodal systems enable a user to provide multiple types of input to communicate with the computing device 1100. The communications interface 1180 generally governs and manages the user input and system output. There is no restriction on operating on any particular hardware arrangement and therefore the basic features here may easily be substituted for improved hardware or firmware arrangements as they are developed.

The technology discussed herein refers to computer-based systems and actions taken by, and information sent to and from, computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

### Methods of Making Marks

The process of creating distinct markings on the seed coat is associated with the fields of plant breeding and plant biotechnology. Plant breeding traditionally involves human interaction to select desired variants among plants based on factors such as yield, quality, case of cultivation, and resistance to pests and environmental stresses. Similar methodologies can be used to produce particular grain varieties having characteristics with specific attributes. The overall process for creating the markings includes breeding the variety of the grain to establish a distinguishable characteristic that is uniform, stable and heritable. The distinguishable characteristic can be established in conjunction with the distinct features being developed in the grain variety, or a developed grain variety can be further bred to impart the distinguishable characteristic.

According to the disclosure, methods of creating a distinguishable characteristic include traditional/conventional plant breeding techniques to create uniform, stable and distinguishable phenotypic seed coat appearance. Traditional methods typically involve the human selection of plants, or groups of plants having the desired characteristic. Methodologies and techniques include, for example, grafting, agro-infiltration, artificial pollination, hybrid breeding, backcrossing, conventional breeding over multiple generations, mutation breeding, mass selection, pedigree methods, and bulk methods.

Biotechnological methodologies can also be utilized to create distinct markings. Steps in a biotechnological methodology can include identifying Quantitative Trait Loci (QTLs) that are highly correlated with unique seed coat color, seed hilum color, and other characteristics. These identified QTLs can then be incorporated into grain breeding programs. Examples of genetic loci controlling soybean seed color and flower pigmentation are shown in Table 1.

TABLE 1
Mapping of Genetic Loci Controlling Soybean Seed Color and Flower Pigmentation
(adapted from Yang et al., “Genetic Analysis of Genes Controlling Natural Variation
of Seed Coat and Flower Colors in Soybean” J. Heredity 2010: 101(6): 757-768.)

| | | | Linkage group | | |
|---|---|---|---|---|---|
| | Locus | | (chromosome | North marker | South marker |
| Gene name | name | Marker name | number) | (cM) [symbol] | (cM) [symbol] |
| AR (anthocyanidin | O | SN013 | A2 (8) | [symbol] (4.1) | S [symbol] 493 (0.0) |
| reductase) | | | | | |
| Invested CHS | [symbol] | SM304, SM305, | A2 (8) | A454.p2 (11 [symbol] ) | GMNOD [symbol] (8.2) |
| repeats | | SM303, SM306 | | | |
| ANS1 | — | SL015, SN014 | B1 (11) | B [symbol] (10.6) | S [symbol] 272 (3.1) |
| ANS2 | — | SN015 | D1a (1) | S [symbol] _414 (11.4) | S [symbol] 129 (2.5) |
| DFR1 | [symbol] | SN016, SM004 | B2 (14) | S [symbol] 287 (5.4) | S [symbol] 467 (1.2) |
| DFR3 | — | SM005, SM325 | D1b (3) | S [symbol] 579 (0.0) | S [symbol] 428 (0.0) |
| DFR2 | [symbol] | SL016 | D2 (2) | S [symbol] 386 (2.2) | S [symbol] 137 (2.8) |
| MYB transcription | [symbol] | SL017 | B2 (14) | S [symbol] (1.9) | S [symbol] 070 (1.9) |
| factor | | | | | |
| F [symbol] H (flavonoid | T | SL305, SN317 | C2 (6) | S [symbol] (7.4) | S [symbol] 365 (0.5) |
| 3′ hydroxylase) | | | | | |
| Anthocyanidin 3-O- | — | SM326, SM327 | D1a (1) | AW7 [symbol] (3.6) | S [symbol] 305 (1.1) |
| glucosyltransferase | | | | | |
| Anthocyanidin 3-O- | — | SM328 | D1b (2) | CA7 [symbol] (4.4) | S [symbol] 558 (6.1) |
| glucosyltransferase | | | | | |
| Anthocyanidin 3-O- | — | SM329 | F (13) | SOYHSP176 (0.0) | N [symbol] SCAR219 (3.3) |
| glucosyltransferase | | | | | |
| Anthocyanidin 3-O- | — | SN017 | J (16) | S [symbol] 11 ( [symbol] ) | S [symbol] 396 (2.1) |
| glucosyltransferase | | | | | |
| Anthocyanidin 3-O- | — | SN018 | J (16) | S [symbol] 11 ( [symbol] ) | S [symbol] 396 (0.4) |
| glucosyltransferase | | | | | |
| F3H1 (flavanone | [symbol] | SN318 | D1b (2) | SL007 (2.0) | S [symbol] 216 (1.7) |
| 3-hydroxylase) | | | | | |
| F3H2 (flavanone | — | SN319 | D1b (2) | SL007 (2.0) | S [symbol] 216 (1.7) |
| 3-hydroxylase) | | | | | |
| F [symbol] (flavano [symbol] | [symbol] | SN019, SL018 | F (13) | S [symbol] 348 (0.0) | S [symbol] 160 (0.0) |
| hydroxylase) | | SM020, SL019 | | | |

[symbol]

indicates data missing or illegible when filed

Biotechnology based plant breeding techniques such as a cisgenesis and intragenesis, embryo culture, chromosome doubling, double haploids, and bridge crossing may be used in the context of the invention. In some biotechnology plant breeding techniques, integrating genomic and/or phenomic platforms may facilitate manipulation of quantitative trait loci (QTL) and/or gene identifications that enable seed improvements or generation of attributes for characteristics. Marker-assisted selection (MAS) allows for the selection of plants/seeds with desirable traits by using DNA markers which are located near a DNA sequence of a desired gene. Because these markers are passed down from one generation to the next, and because they are close to the gene encoding the desired trait, they tend to stay and be passed on together.

Genome editing allows for precise gene manipulation including but not limiting to Clustered regularly interspaced short palindromic repeats (CRISPR)/CRISPR associated protein (Cas) system. For example, plant breeding may be achieved by using CRISPR Cas9 mediated multiplex editing to target and edit multiple genes either by inserting sequences encoding desired traits from DNA templates (e.g., homologous recombination) or silencing genes by introducing indels (e.g., non-homologous end joining). Other gene editing strategies utilize viral vectors through infection techniques with modified plant viruses, and the use of meganucleases, zinc finger nucleases (ZFNs), and transcription-activator like effector nucleases (TALENs). Other methods known in the art for manipulating the epigenic landscape to obtain negative segregants may also be used to modify the genome.

Further aspects of the present disclosure are provided by the subject matter of the following clauses.

A method for tracking an agricultural product variety through a supply chain by identifying the agricultural product variety based on a distinguishable characteristic of a sample wherein the distinguishable characteristic is phenotypic.

The method of the preceding clause, wherein the agricultural product is an ornamental plant, a flower, a shrub, or an agricultural foodstuff.

The method of the preceding clause, wherein the agricultural foodstuff is a fruit, a vegetable, a nut, or a grain.

The method of any preceding clause wherein the distinguishable characteristic is a characteristic selected from a seed characteristic, a plant characteristic or a fruit characteristic.

A method for tracking a variety of a grain through a supply chain by identifying the variety of the grain based on a distinguishable characteristic of a grain sample, wherein the distinguishable characteristic is phenotypic; and the distinguishable characteristic is a seed characteristic of a plurality of seed in the grain sample or a secondary characteristic of plants grown from the variety of the grain in the grain sample.

The method of any preceding clause wherein the distinguishable characteristic is uniform, stable, and heritable.

The method of any preceding clause wherein the seed characteristic is selected from seed shape, seed coat color, hilum color, plant seed luster, and combinations thereof.

The method of any preceding clause wherein identifying comprises a visual assessment.

The method of any preceding clause wherein identifying comprises imaging.

The method of any preceding clause wherein the distinguishable characteristic is obtained through plant breeding or plant biotechnology.

The method of the preceding clause wherein the distinguishable characteristic is obtained by a method including the steps of comprising identifying Quantitative Trait Loci (QTLs) and incorporating the QTLs through plant breeding or plant biotechnology to establish a uniform, stable, and heritable distinguishable characteristic.

The method of any preceding clause wherein identifying is performed by a farmer, grain elevator, grain processor, distributor, retailer, or consumer.

The method of any preceding clause wherein the variety of the grain exhibits a value differentiating characteristic.

The method of the preceding clause wherein the value differentiating characteristic is selected from seed source, a utilization characteristic, a crop management characteristic, disease resistance, drought resistance, pest resistance, herbicide resistance, antioxidant content, nutrient content, improved yield, improved growth, a processing characteristic, an environmental adaptation ability, and combinations thereof.

The method of the preceding clause wherein the utilization characteristic is selected from the group consisting of human consumption, animal consumption, oil source, solid food source, genetically modified origin, non-genetically modified origin, geographic origin, and combinations thereof or the crop management characteristic is selected from the group consisting of agronomic management, disease resistance, antioxidant content, nutrient content, improved yield, improved growth, a processing characteristic, a utilization characteristic, an environmental adaptation ability, and combinations thereof.

The method according to any preceding clause, further comprising segregating the variety of the grain from a different variety of the grain.

The method according to the preceding clause, wherein the different variety of the grain is a commodity grain or a variety of the grain with a different value differentiating property.

The method of any preceding clause further comprising managing agricultural production, for example grain production, for the variety of the agricultural product.

The method of the preceding clause wherein managing agricultural production, for example grain production, is selected from one or more of agronomic management, varying plant density, applying fertilizer, applying herbicide, applying pesticide, varying moisture conditions, varying crop location, selecting cultivation methods, and selecting harvesting methods.

The method of any preceding clause wherein the secondary characteristic is selected from flower color, leaf shape, pubescence, pod appearance, plant shape, stem termination, bloom habit, growth habitat, leaf habitat, and combinations thereof.

The method of the preceding clause wherein the secondary characteristic comprises a feature allowing identification by imaging.

The method of the preceding clause wherein the secondary characteristic comprises a color or pigmentation detectable by multispectral imaging, hyperspectral imaging, or LiDAR.

The method of any preceding clause wherein the agricultural product is a grain selected from the group consisting of soybeans, corn, rye, rice, wheat, buckwheat, oats, millet, and barley.

A method of making an agricultural product variety identifiable by a distinguishable characteristic by breeding the agricultural product variety to establish a distinguishable characteristic that is uniform, stable and heritable.

The method of making an agricultural product variety identifiable of the preceding clause, wherein the is an ornamental plant, a flower, a shrub, or an agricultural foodstuff.

The method of making an agricultural product variety identifiable the preceding clause, wherein the agricultural foodstuff is a fruit, a vegetable, a nut, or a grain.

A method of making a variety of a grain identifiable by a distinguishable characteristic by breeding the variety of the grain to establish a distinguishable characteristic that is uniform, stable and heritable, wherein the distinguishable characteristic is (i) a seed characteristic of the variety of the grain or (ii) a secondary characteristic of plants grown from the variety of the grain.

The method of the preceding clause wherein the seed characteristic is selected from seed shape, seed coat color, hilum color, plant seed luster, and combinations thereof.

The method of the preceding clause for making a variety of a grain identifiable wherein the distinguishable secondary characteristic is selected from flower color, leaf shape, pubescence, mature pod appearance, bloom habit, color or pigmentation detectable by multispectral imaging or hyperspectral imaging.

The method of any preceding clause for making a variety of a grain identifiable wherein the breeding is inbreeding or hydridization.

The method of any preceding clause for making a variety of a grain identifiable further comprising identifying Quantitative Trait Loci (QTLs) associated with the distinguishable characteristic, and incorporating the QTLs into the grain.

The method of the preceding clause for making a variety of a grain identifiable, wherein the QTLs are incorporated through plant breeding or plant biotechnology.

The method of any preceding clause for making a variety of a grain identifiable wherein the grain is a grain seed or a grain plant.

A taxonomy system for identifying an agricultural product variety by defining a set of distinguishable structural features of the an agricultural product variety, wherein the distinguishable structural features are selected from seed characteristics and plant characteristics.

The taxonomy system of the preceding clause wherein the agricultural product variety is a grain variety and the distinguishable structural features are selected from the group consisting of seed shape, seed coat color, hilum color, plant seed luster, flower color, leaf shape, pubescence, pod appearance, plant shape, stem termination, bloom habit, leaf habitat, and combinations thereof.

The taxonomy system of any preceding clause wherein the set of distinguishable structural features are hierarchical.

The taxonomy system of any preceding clause wherein the agricultural product variety comprises a value differentiating characteristic.

The taxonomy system of the preceding clause wherein the value differentiating characteristic is selected from seed source, a utilization characteristic, a crop management characteristic, disease resistance, drought resistance, pest resistance, herbicide resistance, antioxidant content, nutrient content, improved yield, improved growth, a processing characteristic, an environmental adaptation ability, and combinations thereof.

The taxonomy system of the preceding clause wherein the utilization characteristic is selected from the group consisting of human consumption, animal consumption, oil source, solid food source, genetically modified origin, non-genetically modified origin, geographic origin, and combinations thereof or the crop management characteristic is selected from the group consisting of agronomic management, disease resistance, antioxidant content, nutrient content, improved yield, improved growth, a processing characteristic, a utilization characteristic, an environmental adaptation ability, and combinations thereof.

The method or system of any preceding clause wherein the seed shape is selected from the group consisting of spherical rounded, spherical flattened, elongate and elongate flattened.

The method or system of any preceding clause wherein the seed coat color is selected from the group consisting of yellow, clack saddle, clack, brown, black spots, black rings, dark brown, red-brown, buff, dichromate and green.

The method or system of any preceding clause wherein the hilum color is selected from the group consisting of black, imperfect black, grey, dark brown, brown, light brown, imperfect yellow, and yellow.

The method or system of any preceding clause wherein the plant seed luster is selected from the group consisting of dull, mudfilm, mud-free-film and lustrous.

The method or system of any preceding clause wherein the flower color is selected from the group consisting of white, purple, and other.

The method or system of any preceding clause wherein the leaf shape is selected from the group consisting of lanceolate, triangular, pointed ovate, rounded ovate and linear.

The method or system of any preceding clause wherein the pod appearance is selected from the group consisting of brown, dark brown and black.

The method or system of any preceding clause wherein the plant shape is selected from the group consisting of erect, semi-erect, indeterminate, semi-prostrate, prostrate, wild, semi-wild, and combinations thereof.

The method or system of any preceding clause wherein the stem termination is selected from the group consisting of determinate and indeterminate.

The method or system of any preceding clause wherein the grain is selected from the group consisting of soybeans, corn rye, rice, wheat, buckwheat, oats, millet, and barley.

The taxonomy system of any preceding clause wherein the taxonomy system identifies one or more of grain quality, grain origin, grain ownership, geographic origin, and regulatory status.

The taxonomy system of any preceding clause wherein the grain variety is identifiable by visual assessment.

The method or system of any preceding clause wherein the grain variety is identifiable by imaging.

The embodiments illustrated and discussed in this disclosure are intended only to teach those skilled in the art the best way known to the inventors to make and use the disclosed methods, compositions and systems. Nothing in this disclosure should be considered as limiting the scope of the present invention. All examples presented are representative and non-limiting. The above-described features may be modified or varied, without departing from the invention, as appreciated by those skilled in the art in light of the above teachings. It is therefore to be understood that, within the scope of any claims supported by the disclosure and their equivalents, the invention may be practiced other than as specifically described.

1. A method for tracking a variety of a grain through a supply chain comprising:
obtaining the variety of grain from a seed producer, wherein the variety of the grain has a distinguishable characteristic that differentiates the grain variety from other varieties of a grain; and
(a) the distinguishable characteristic is phenotypic;
(b) the distinguishable characteristic is
(i) a seed characteristic of a plurality of seed in the grain sample;
(ii) the seed characteristic is a visual characteristic; and
(iii) the seed characteristic is a unique seed marking; and
identifying the variety of the grain in the supply chain;
wherein the identifying is performed by at least one of:
a seed producer to track the grain within the seed breeding program or tracking the grain variety end-to-end agriculture commodity supply chains,
a farmer to identify the purchase of a proper seed variety, to verify the authenticity of the grain variety in a purchased grain, or for crop management of the grain variety,
a grain elevator to assure the authenticity of a grain variety, or segregate and store the grain variety from other grain varieties,
a grain processor to assure the authenticity of a grain variety, segregate and store the grain variety from other grain varieties, or more effectively market value-added end products, and
a distributor to produce properly identified end products and market authenticated consumer-packaged goods to a retailer or to verify the authenticity of the grain variety in a purchased grain.

2. The method of claim 1, wherein the distinguishable characteristic is uniform, stable, and heritable.

3. The method of claim 1, wherein the seed characteristic is selected from seed shape, seed coat color, hilum color, plant seed luster, and combinations thereof.

4. The method of claim 3, wherein the seed shape is selected from the group consisting of spherical rounded, spherical flattened, elongate and elongate flattened or
the seed coat color is selected from the group consisting of yellow, clack saddle, clack, brown, black spots, black rings, dark brown, red-brown, buff, dichromate and green, or
the hilum color is selected from the group consisting of black, imperfect black, grey, dark brown, brown, light brown, imperfect yellow, and yellow, or
the plant seed luster is selected from the group consisting of dull, mudfilm, mud-free-film and lustrous.

5. The method of claim 1, wherein identifying comprises a visual assessment or imaging.

6. The method of claim 1, wherein the seed producer produces the distinguishable characteristic through plant breeding or plant biotechnology.

7. The method of claim 1, wherein the distinguishable characteristic is obtained by a method comprising
identifying Quantitative Trait Loci (QTLs) and
incorporating the QTLs through plant breeding or plant biotechnology to establish a uniform, stable, and heritable distinguishable characteristic.

8. (canceled)

9. The method of claim 1, wherein the variety of the grain exhibits a value differentiating characteristic.

10. The method of claim 9, wherein
the value differentiating characteristic is selected from seed source, a utilization characteristic, a crop management characteristic, disease resistance, drought resistance, pest resistance, herbicide resistance, antioxidant content, nutrient content, improved yield, improved growth, a processing characteristic, an environmental adaptation ability, and combinations thereof; and wherein
the utilization characteristic is selected from the group consisting of human consumption, animal consumption, oil source, solid food source, genetically modified origin, non-genetically modified origin, geographic origin, and combinations thereof and/or
the crop management characteristic is selected from the group consisting of agronomic management, disease resistance, antioxidant content, nutrient content, improved yield, improved growth, a processing characteristic, a utilization characteristic, an environmental adaptation ability, and combinations thereof.

11. The method of claim 1, further comprising managing grain production for the variety of the grain;
wherein managing grain production is selected from one or more of agronomic management, varying plant density, applying fertilizer, applying herbicide, applying pesticide, varying moisture conditions, varying crop location, selecting cultivation methods, and selecting harvesting methods.

12. (canceled)

13. The method of claim 1, wherein the grain is selected from the group consisting of soybeans, corn, rye, rice, wheat, buckwheat, oats, millet, and barley.

14. A method of making a variety of a grain identifiable by a distinguishable characteristic comprising,
breeding the variety of the grain to establish a distinguishable characteristic that is uniform, stable and heritable and differentiates the grain variety from other varieties of the grain; wherein the distinguishable characteristic is (i) a seed characteristic of the variety of the grain (ii) a secondary characteristic of plants grown from the variety of the grain in the grain sample or (iii) a combination thereof; and
wherein the seed characteristic is a visual characteristic, and the seed characteristic is a unique seed marking, and
the breeding comprises plant breeding or plant biotechnology.

15. The method of claim 14, further comprising identifying Quantitative Trait Loci (QTLs) associated with the distinguishable characteristic, and
incorporating the QTLs into the grain; wherein the QTLs are incorporated through plant breeding or plant biotechnology.

16. A grain produced by the method of claim 14, wherein the grain is a grain seed or a grain plant.

17. A taxonomy system for identifying a grain variety comprising defining a set of distinguishable structural features of the grain variety, wherein the distinguishable structural features are selected from the group consisting of seed shape, seed coat color, hilum color, plant seed luster, flower color, leaf shape, pubescence, pod appearance, plant shape, stem termination, bloom habit, leaf habitat, and combinations thereof.

18. The taxonomy system of claim 17, wherein the set of distinguishable structural features are hierarchical.

19. The taxonomy system of claim 17, wherein the taxonomy system identifies one or more of grain quality, grain origin, grain ownership, geographic origin, and regulatory status.

20. The taxonomy system of claim 17, wherein the grain variety is identifiable by visual assessment or imaging.

21. The method of claim 1, wherein the identifying is performed by at least one of
a farmer to verify the authenticity of the grain variety,
a grain elevator to segregate the grain variety from other grain varieties in storage to maintain identity preservation and sell and deliver the identity preserved grain to customers,
a grain processor to authenticate the identity of the grain variety, segregate the grain variety in storage to maintain identify preservation, perform value added grain processing on the grain variety, segregate end products of the grain processing, market end products identifies as the grain variety,
a distributor to produce consumer-packaged goods using the grain variety purchased from the grain processor.

22. A method for tracking a variety of a grain through a supply chain comprising:
obtaining the variety of grain from a seed producer, wherein the variety of the grain has a distinguishable characteristic that differentiates the grain variety from other varieties of a grain; and
(a) the distinguishable characteristic is phenotypic;
(b) the distinguishable characteristic is a secondary characteristic of plants grown from the variety of the grain in the grain sample; and
identifying the variety of the grain in the supply chain;
wherein the identifying is performed by at least one of
a farmer or distributor to verify the authenticity of the grain variety in a purchased grain,
a grain elevator or a grain processor to in assure the authenticity of a grain variety, and/or segregate and store the grain variety from other grain varieties.

23. The method of claim 22, wherein the secondary characteristic is selected from pigmentation, flower color, leaf shape, pubescence, pod appearance, plant shape, stem termination, bloom habit, growth habitat, leaf habitat, and combinations thereof.

24. The method of claim 23, wherein
the flower color is selected from the group consisting of white, purple, and other, or
the leaf shape is selected from the group consisting of lanceolate, triangular, pointed ovate, rounded ovate and linear, or
the pod appearance is selected from the group consisting of brown, dark brown and black, or
the plant shape is selected from the group consisting of erect, semi-erect, indeterminate, semi-prostrate, prostrate, wild, semi-wild, and combinations thereof, and/or
the stem termination is selected from the group consisting of determinate and indeterminate.

25. The method of claim 22, wherein identifying comprises a visual assessment or imaging.

26. The method of claim 22, wherein the seed producer produces the distinguishable characteristic through plant breeding or plant biotechnology.

27. The method of claim 22, wherein the distinguishable characteristic is obtained by a method comprising
identifying Quantitative Trait Loci (QTLs) and
incorporating the QTLs through plant breeding or plant biotechnology to establish a uniform, stable, and heritable distinguishable characteristic.

28. The method of claim 22, wherein the variety of the grain exhibits a value differentiating characteristic selected from seed source, a utilization characteristic, a crop management characteristic, disease resistance, drought resistance, pest resistance, herbicide resistance, antioxidant content, nutrient content, improved yield, improved growth, a processing characteristic, an environmental adaptation ability, and combinations thereof; and wherein
the utilization characteristic is selected from the group consisting of human consumption, animal consumption, oil source, solid food source, genetically modified origin, non-genetically modified origin, geographic origin, and combinations thereof and/or
the crop management characteristic is selected from the group consisting of agronomic management, disease resistance, antioxidant content, nutrient content, improved yield, improved growth, a processing characteristic, a utilization characteristic, an environmental adaptation ability, and combinations thereof.

29. The method of claim 22, further comprising managing grain production for the variety of the grain;
wherein managing grain production is selected from one or more of agronomic management, varying plant density, applying fertilizer, applying herbicide, applying pesticide, varying moisture conditions, varying crop location, selecting cultivation methods, and selecting harvesting methods.

30. The method of claim 22, wherein the secondary characteristic comprises a color or pigmentation detectable by multispectral imaging, hyperspectral imaging, or LiDAR.

31. The method of claim 22, wherein the grain is selected from the group consisting of soybeans, corn, rye, rice, wheat, buckwheat, oats, millet, and barley.
